(19) United States
(12) Patent Application Publication
(10) Pub. No.: US 2026/0314615 A1
(43) Pub. Date: Oct. 15, 2026

(54) SMOKER OVEN

(71) Applicant: Ningbo Lemeijia Electric Equipment Technology Co., Ltd., Ningbo (CN)

(72) Inventor: Qifeng LUO, Ningbo (CN)

(73) Assignee: Ningbo Lemeijia Electric Equipment Technology Co., Ltd., Ningbo (CN)

(21) Appl. No.: 19/314,304

(22) Filed: Aug. 29, 2025

(30) Foreign Application Priority Data
Apr. 14, 2025 (CN) 202510460285.5
Jul. 8, 2025 (CN) 202510935171.1

(51) Int. Cl. A23B 4/056; A23B 4/052

(52) U.S. Cl. CPC A23B 4/056; A23B 4/0523

(57) ABSTRACT

A smoker oven includes a smoking assembly, an inner container and a box body. The smoking assembly and the inner container are arranged in the box body; a side of the inner container close to a box door is provided with a cooking opening, and a side of the inner container close to the smoking assembly is provided with a smoke outlet. The smoking assembly includes a material barrel, a feeder, a heater, a flue and a water tank cavity; the feeder is arranged below the material barrel; a feeding roller and a baffle are arranged in the feeder; the heater is arranged below the feeder; the water tank cavity is arranged below the heater; a water tank is arranged in the water tank cavity.

### CROSS-REFERENCE TO RELATED APPLICATIONS

This application claims priority to Chinese Patent Application No. 202510460285.5, filed on Apr. 14, 2025, and Chinese Patent Application No. 202510935171.1, filed on Jul. 8, 2025. All of the aforementioned applications are incorporated herein by reference in their entireties.

### TECHNICAL FIELD

The present application relates to the technical field of household electrical appliances, in particular to a smoker oven.

### BACKGROUND

A smoker oven is a kitchen device that combines oven and smoking functions. The smoker oven is mainly used to add a smoky flavor to food during a cooking process. Through low-temperature slow roasting and smoke smoking, meat, fish, vegetables and other food ingredients absorb fragrance produced by burning of wood chips, whereby a unique flavor and taste are formed. At present, conventional smoker ovens on the market mostly use a method of directly heating all smoking materials. The amount of smoke in the oven is controlled by adjusting the size of a flue outlet. This method not only consumes a lot of wood chips and has a low utilization rate, but may also generate excess smoke due to insufficient combustion, resulting in resource waste and environmental hazard.

### SUMMARY

In view of defects existing in the existing smoker ovens, the present application aims to solve the technical problem of providing a smoker oven which quantitatively heats smoking wood chips, controls the amount of smoke generated by smoking wood chips by adjusting the temperature of a heater. In this way, the amount of smoke in an inner container is adjusted; and the burned-off wood chips enter a water tank to be extinguished. The smoking wood chips are fully utilized, thereby avoiding resource waste and eliminating environmental hazard.

In order to achieve the above object, according to one aspect of the present application, the present application is implemented by the following technical measures: a smoker oven includes a smoking assembly, an inner container and a box body; the smoking assembly and the inner container are arranged in the box body; one side of the box body is provided with a box door, a material barrel opening and a water tank opening; a side of the inner container close to the box door is provided with a cooking opening, and a side of the inner container close to the smoking assembly is provided with a smoke outlet; the smoking assembly includes a material barrel, a feeder, a heater, a flue and a water tank cavity; the feeder is arranged below the material barrel; a quantitative feeding device is arranged in the feeder; the heater is arranged below the feeder; the water tank cavity is arranged below the heater; a water tank is arranged in the water tank cavity; and the flue covers above the water tank cavity to communicate the water tank cavity with the smoke outlet.

Further, the quantitative feeding device includes a feeding roller and a baffle; the top of the feeder is provided with a second feeding opening; the bottom of the feeder is provided with a second discharge opening; the feeding roller and the baffle are arranged at the second discharge opening; the bottom of the second discharge opening is provided with the heater; the baffle and the feeding roller press against each other to block the second discharge opening; and a feeding groove is arranged on the feeding roller.

Further, the heater is provided with a third feeding opening and a third discharge opening; and the heater is further provided with a discharge device for controlling quantitative drop of wood chips.

Further, the discharge device is a pushing plug; the pushing plug is arranged in the heater; the pushing plug is connected to the feeding roller through an eccentric connecting rod; rotation of the feeding roller can drive the pushing plug to move in the heater; and a heating sheet is further arranged in the heater.

Further, the discharge device includes a transmission assembly and a switch plate; both a top end and a bottom end of the heater are equipped with an opening; the switch plate is hinged with the bottom of the heater; a torsion spring is arranged at a hinge part; one side of the switch plate extends outward to form a connecting rod; one side of the feeding roller is provided with the transmission assembly; the feeding roller rotates to control opening and closing of the switch plate through the transmission assembly; the transmission assembly includes a cam, a transmission rod, a transmission frame and a micro switch; the cam is coaxial with the feeding roller; a cam tip corresponds to a position of the feeding groove; a contact block is arranged on an outer side of a cam base circle; the micro switch is arranged on an outer side of the feeder; the transmission rod is inserted into the transmission frame; the top of the transmission rod presses against the cam; the transmission rod is sleeved with a second spring; one end of the second spring presses against the transmission rod, and the other end presses against the transmission frame; and one end of the transmission rod extends outward and presses against the connecting rod of the switch plate.

Further, the transmission rod is in the shape of "n"; the second spring is sleeved on each vertical rod of the transmission rod; and one vertical rod of the transmission rod extends horizontally outward and presses against the connecting rod of the switch plate.

Further, the material barrel is arranged in a material barrel cavity; the material barrel is provided with a material groove; one side of the material barrel cavity is provided with a first feeding opening; the bottom of the material barrel cavity is provided with a first discharge opening; and the first feeding opening and the material barrel opening face each other.

Further, both a top end and a bottom end of the material groove are equipped with an opening; a sliding plate is movably connected at a bottom opening; and a baffle plate is arranged on one side of the second feeding opening close to the material barrel opening.

Further, both a top end and a bottom end of the material groove are equipped with an opening, a sliding plate is movably connected at a bottom opening; a baffle plate is arranged on one side of the first discharge opening close to the first feeding opening; the bottom of the material barrel is provided with a sliding rod; the sliding plate is movably connected to the sliding rod; a first spring is arranged on the sliding rod; one end of the first spring presses against the sliding plate, and the other end presses against the material barrel; and when the first spring is in a natural state, the sliding plate seals the bottom of the material groove.

Further, the top of the water tank cavity is provided with a fourth feeding opening; one side of the water tank cavity is provided with a fourth discharge opening; the fourth discharge opening and the water tank opening face each other; a water tank seat is arranged in the water tank cavity; and the water tank is arranged in the water tank seat.

Compared with the prior art, the present application has the advantages that: in the smoker oven, the feeding roller and the baffle are arranged in the feeder; the feeding roller and the baffle cooperate to control the amount of wood chips entering the heater for heating; the feeding roller rotates intermittently; the smoking wood chips are quantitatively heated through the cooperation of the feeding roller and the discharge device; and the amount of smoke generated by the smoking wood chips is controlled by adjusting the temperature of the heater. In this way, the amount of smoke in the inner container is adjusted; and the burned-off wood chips enter the water tank to be extinguished. The smoking wood chips are fully utilized, thereby avoiding resource waste and eliminating environmental hazard.

### BRIEF DESCRIPTION OF THE DRAWINGS

The accompanying drawings, which form a part of the present application, are intended to provide a further understanding of the present application, and the schematic embodiments of the present application and the description thereof are intended to explain the present application, and do not constitute an undue limitation of the present application. In the drawings:

FIG. 1 is an exploded structural schematic diagram of a smoker oven according to embodiment 1 of the present application;

FIG. 2 is a schematic structural diagram of a material barrel cavity and a material barrel according to embodiment 1 of the present application;

FIG. 3 is a schematic structural diagram of a feeder according to embodiment 1 of the present application;

FIG. 4 is a schematic structural diagram of a feeder, a heater, and a flue according to embodiment 1 of the present application;

FIG. 5 is a schematic structural diagram of a water tank cavity and a water tank seat according to the present application;

FIG. 6 is an exploded structural schematic diagram of a smoker oven according to embodiment 2 of the present application;

FIG. 7 is a schematic structural diagram of a material barrel cavity and a material barrel according to embodiment 2 of the present application;

FIG. 8 is a schematic structural diagram of a bottom of a material barrel according to embodiment 2 of the present application; and

FIG. 9 is a schematic structural diagram of a feeder, a heater, and a transmission assembly according to embodiment 2 of the present application.

### DETAILED DESCRIPTION OF THE EMBODIMENTS

Hereinafter, the present application will be described in detail with reference to the accompanying drawings in conjunction with embodiments. It should be noted that the embodiments and features in the embodiments in the present application may be combined with each other as long as there is no conflict.

In the description of the present application, it should be understood that the orientation or positional relationship indicated by the terms "center", "upper", "lower", "left", "right", "vertical", "horizontal", "inner", "outer", "bottom", "top", etc. is based on the orientation or positional relationship shown in the accompanying drawings, and is merely intended to facilitate and simplify the description of the present application, and does not indicate or imply that the referred device or element must have a specific orientation, be constructed and operate in a specific orientation, and therefore cannot be understood as a limitation of the present application.

### Embodiment 1

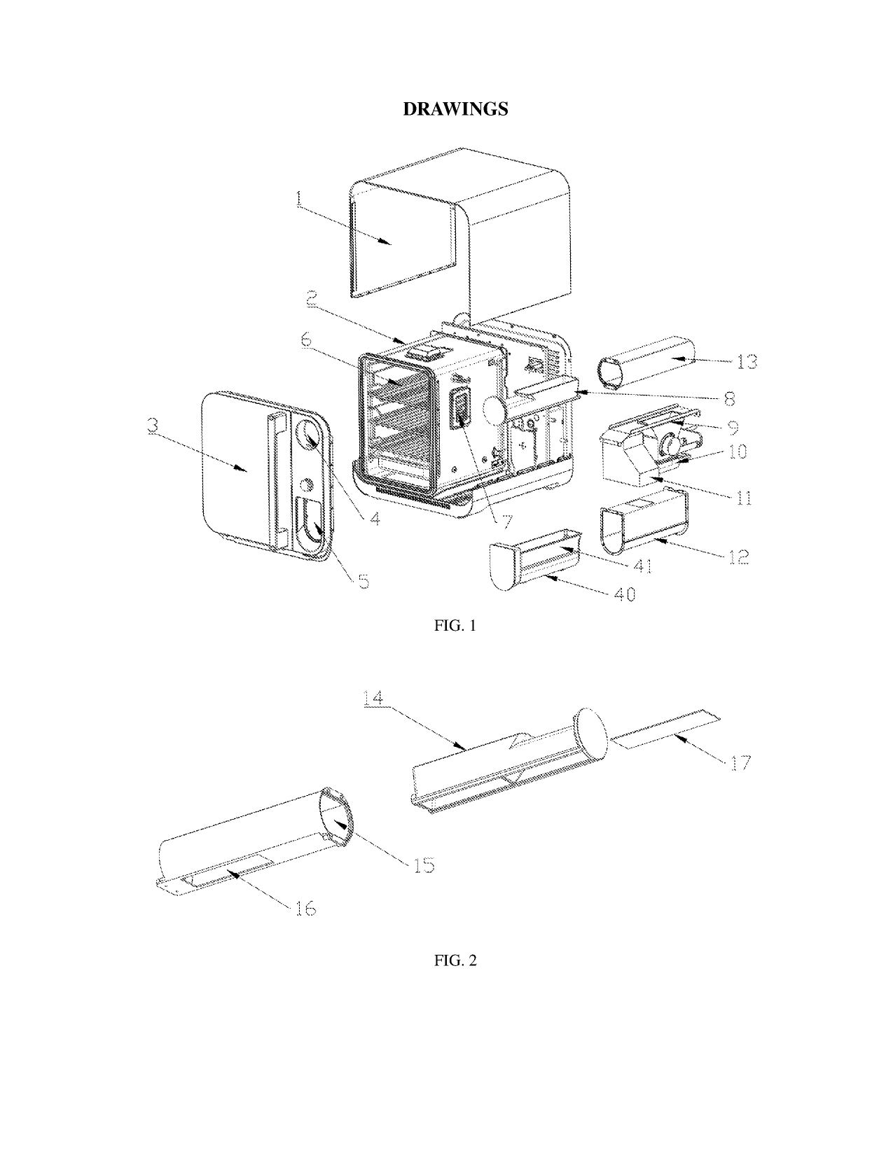

Referring to FIG. 1, this embodiment provides a smoker oven, including a smoking assembly, an inner container 2, and a box body 1. The smoking assembly and the inner container 2 are arranged in the box body 1; one side of the box body 1 is provided with a box door 3, a material barrel opening 4, and a water tank opening 5; a side of the inner container 2 close to the box door 3 is provided with a cooking opening 6, and a side of the inner container 2 close to the smoking assembly is provided with a smoke outlet 7; and the smoking assembly includes a material barrel cavity 13, a material barrel 8, a feeder 9, a heater 10, a flue 11, a water tank cavity 12, and a water tank seat 40.

Continuing to explain in combination with FIG. 2, one side of the material barrel cavity 13 is provided with a first feeding opening 15; the bottom of the material barrel cavity 13 is provided with a first discharge opening 16; the first feeding opening 15 and the material barrel opening 4 face each other; the material barrel 8 is arranged in the material barrel cavity 13; the material barrel 8 is provided with a material groove 14; both a top end and a bottom end of the material groove 14 are equipped with an opening; a bottom opening is movably connected to a sliding plate 17; and the sliding plate 17 can block the bottom opening.

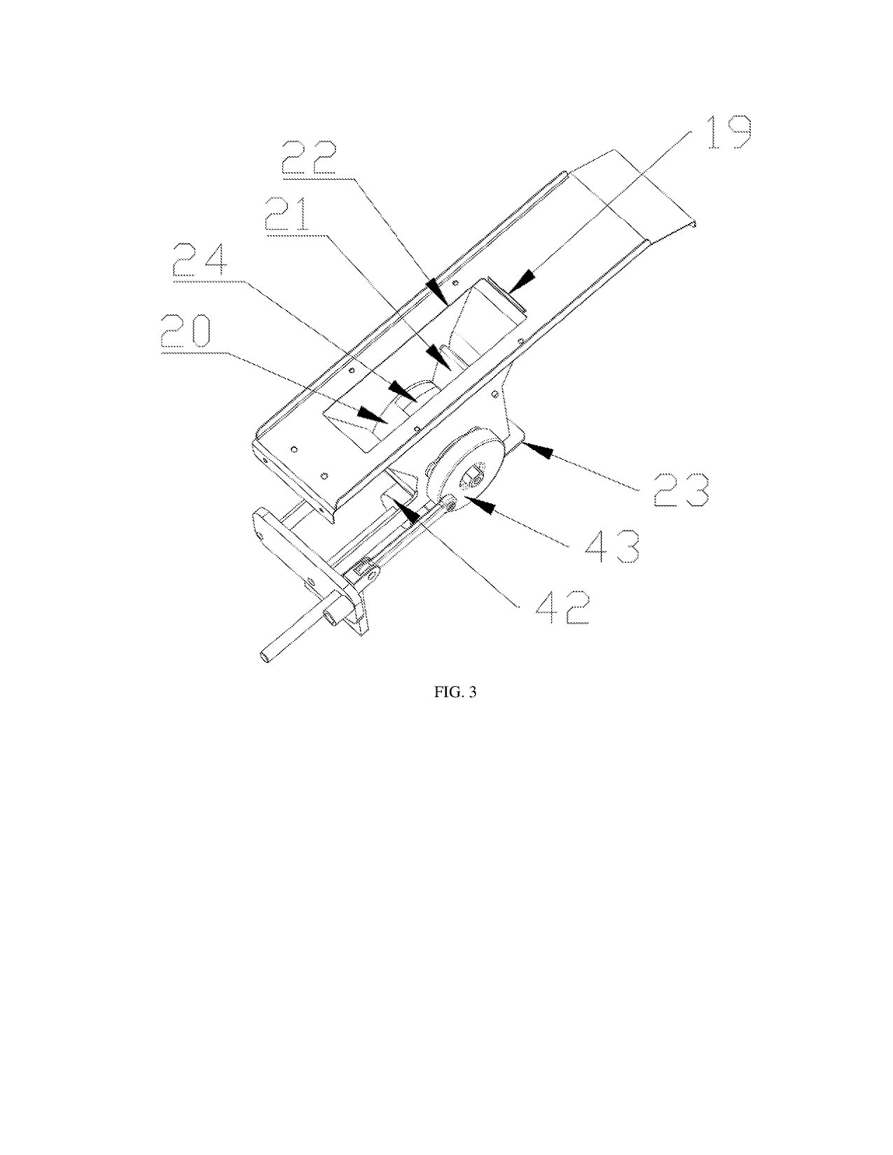

Continuing to explain in combination with FIG. 3, the top of the feeder 9 is provided with a second feeding opening 22; a baffle plate 19 is arranged on one side of the second feeding opening 22 close to the material barrel opening 4; the bottom of the feeder 9 is provided with a second discharge opening 23; a feeding roller 20 and a baffle 21 are arranged at the second discharge opening 23; the baffle 21 and the feeding roller 20 press against each other to block the second discharge opening 23; and a feeding groove 24 is arranged on the feeding roller 20.

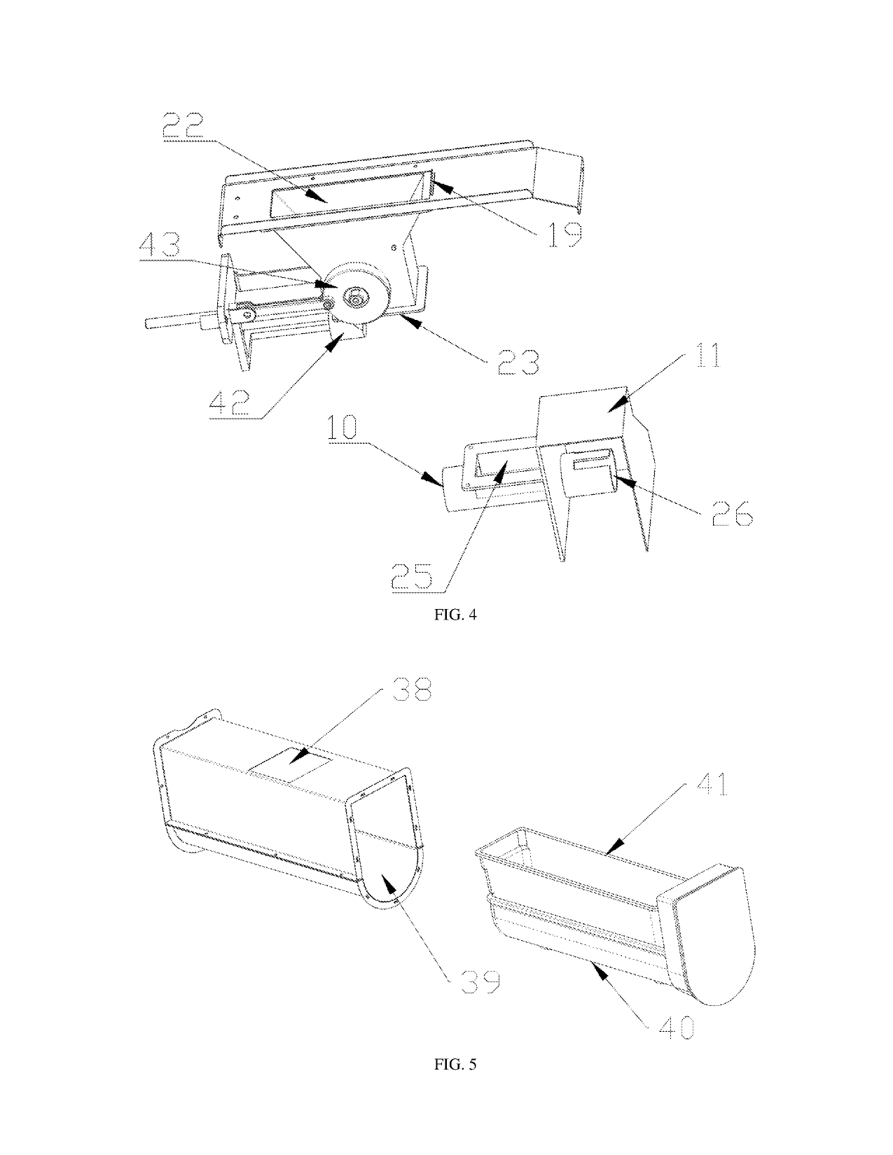

Continuing to explain in combination with FIG. 4, the bottom of the second discharge opening 23 is provided with the heater 10; the top of the heater 10 is provided with a third feeding opening 25; one side of the heater 10 is provided with a third discharge opening 26; the bottom close to the third discharge opening 26 is provided with a heating sheet; a pushing plug 42 is arranged in the heater 10; the pushing plug 42 is connected to the feeding roller 20 through an eccentric connecting rod 43; and rotation of the feeding roller 20 can drive the pushing plug 42 to move in the heater 10.

Continuing to explain in combination with FIG. 5, the top of the water tank cavity 12 is provided with a fourth feeding opening 38; one side of the water tank cavity 12 is provided with a fourth discharge opening 39; the fourth discharge opening 39 and the water tank opening 5 face each other; the water tank seat 40 is arranged in the water tank cavity 12; a the water tank 41 is arranged in the water tank seat 40; and the flue 11 covers above the water tank cavity 12 to communicate the water tank cavity 12 with the smoke outlet 7.

During use of the smoker oven provided by this embodiment, the sliding plate 17 on the bottom of the material barrel 8 blocks the bottom of the material groove 14; smoking wood chips are put into the material groove 14; the material barrel 8 is pushed from the material barrel opening 4 into the material barrel cavity 13; during pushing, the baffle plate 19 presses against the sliding plate 17 to push the sliding plate 17 away; the smoking wood chips in the material groove 14 enter the feeder 9 from the material groove 14 via the first discharge opening 16 and the second feeding opening 22; after the water tank 41 is filled with water, the water tank 41 is put on the water tank seat 40; the water tank seat 40 is pushed from the water tank opening 5 into the water tank cavity 12; the smoking assembly is started; the feeding roller 20 rotates; during rotation, the smoking wood chips enter the feeding groove 24, are rotated with the feeding groove 24 to reach a position above the third feeding opening 25, and drop into the heater 10 via the third feeding opening 25; the remaining smoking wood chips are stopped by the baffle 21 to still stay in the feeding groove 24; the pushing plug 42 is driven by the eccentric connecting rod 43 to push the wood chips to the third discharge opening 26; the feeding roller 20 stops rotation, that is, feeding into the heater 10 is stopped; the heating sheet heats the wood chips at the third discharge opening 26; the smoking wood chips after heating generate smoke which is guided by the flue 11 to enter the inner container 2 from the smoke outlet 7 for food smoking; a user can control the amount of smoke of the wood chips by adjusting the temperature of the heating sheet in the heater 10; after the heated wood chips are completely burned off, the feeding roller 20 rotates again; the smoking wood chips enter the heater 10 from the feeder 9 again, and are then pushed by the pushing plug 42 to press against the burned-off smoking wood chips; the burned-off smoking wood chips drop into the water tank 41 from the fourth feeding opening 38; and the burned-off smoking wood chips are extinguished after touching the water. The smoking wood chips are quantitatively heated through the cooperation of the feeding roller 20, the baffle 21 and the pushing plug 42. The smoking wood chips are fully utilized, thereby avoiding resource waste and eliminating environmental hazard.

### Embodiment 2

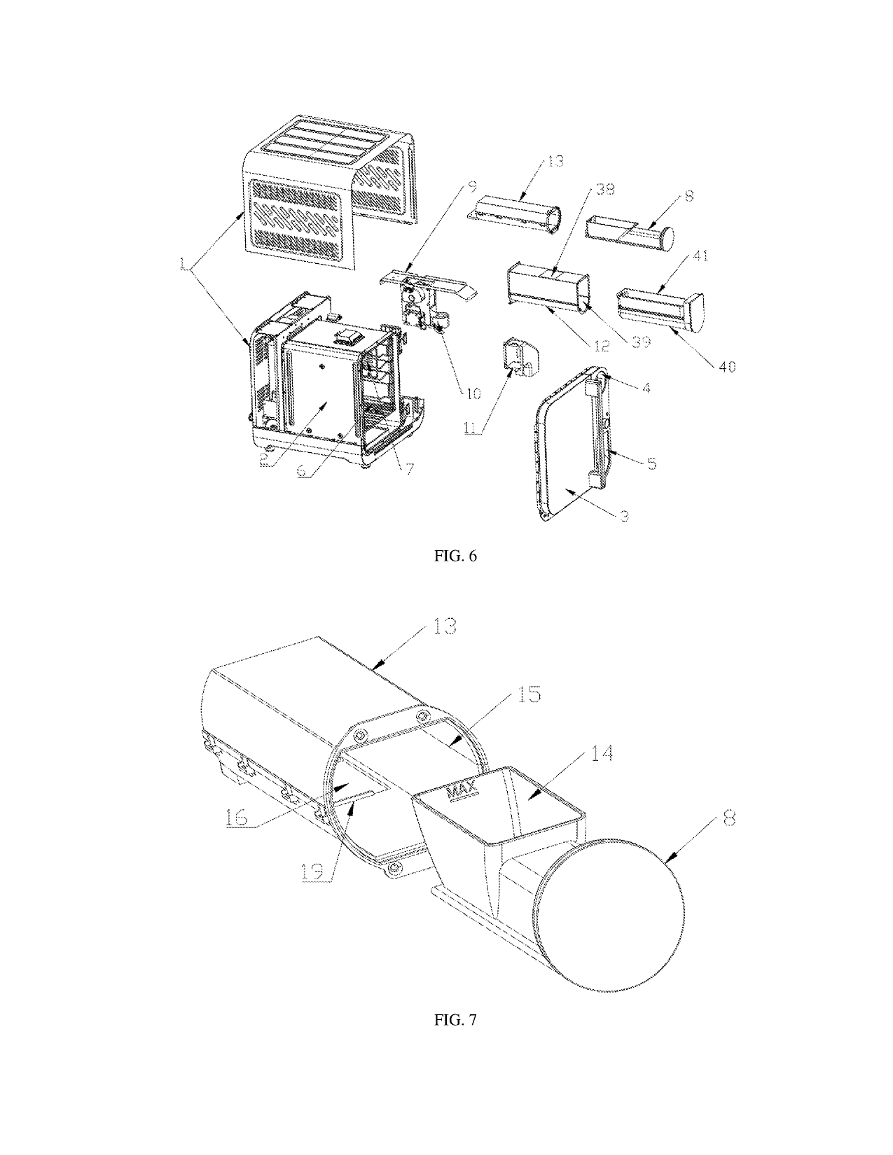

Referring to FIG. 6, a smoker oven provided by this embodiment includes a smoking assembly, an inner container 2 and a box body 1; the smoking assembly and the inner container 2 are arranged in the box body 1; one side of the box body 1 is provided with a box door 3, a material barrel opening 4 and a water tank opening 5; a side of the inner container 2 close to the box door 3 is provided with a cooking opening 6, and a side of the inner container 2 close to the smoking assembly is provided with a smoke outlet 7; the smoking assembly includes a material barrel 8, a feeder 9, a heater 10, a flue 11 and a water tank cavity 12; the feeder 9 is arranged below the material barrel 8; a quantitative feeding device is arranged in the feeder 9; the heater 10 is arranged below the feeder 9; the water tank cavity 12 is arranged below the heater 10; the top of the water tank cavity 12 is provided with a fourth feeding opening 38; one side of the water tank cavity 12 is provided with a fourth discharge opening 39; the fourth discharge opening 39 and the water tank opening 5 face each other; a water tank seat 40 is arranged in the water tank cavity 12; a water tank 41 is arranged in the water tank seat 40; and the flue 11 covers above the water tank cavity 12 to communicate the water tank cavity 12 with the smoke outlet 7.

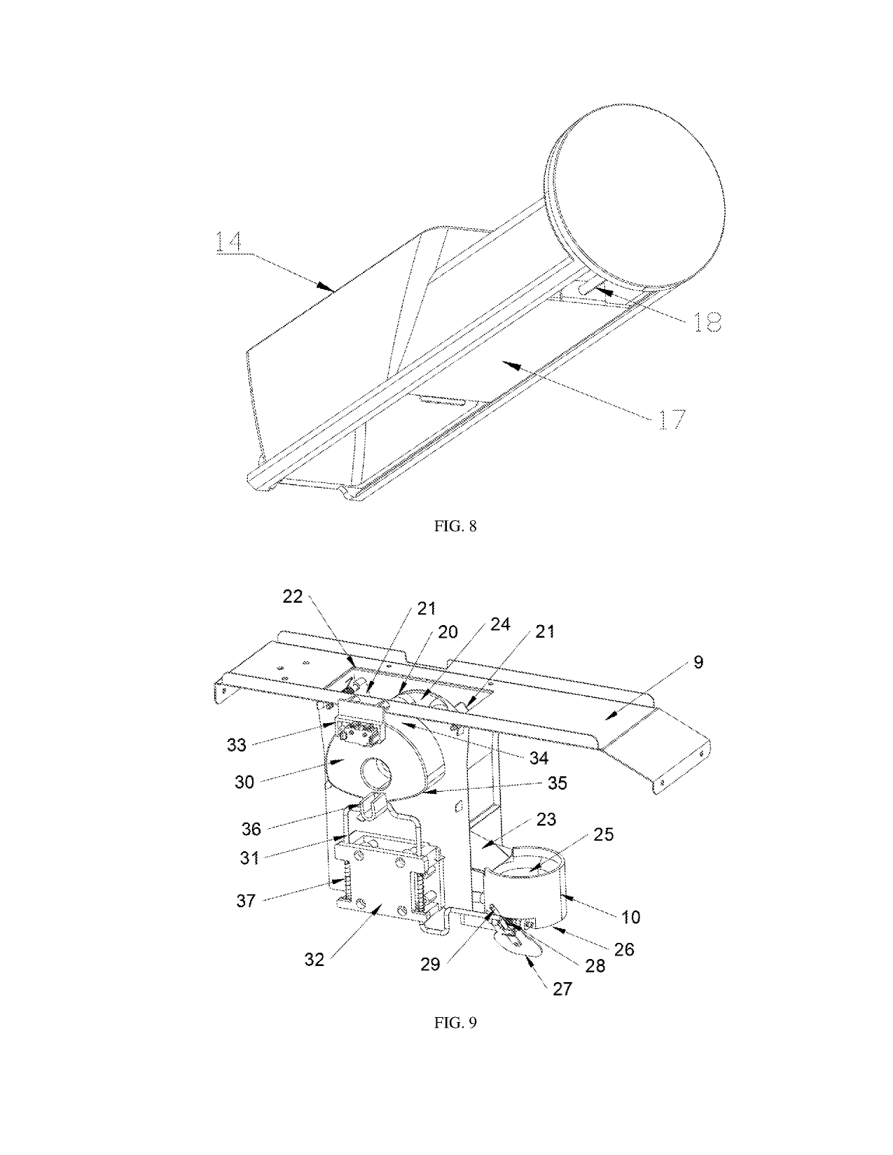

Continuing to explain in combination with FIGS. 7 and 8, the material barrel 8 is arranged in the material barrel cavity 13; the material barrel 8 is provided with a material groove 14; one side of the material barrel cavity 13 is provided with a first feeding opening 15, and the bottom is provided with a first discharge opening 16; the first feeding opening 15 and the material barrel opening 4 face each other; one side of the first discharge opening 16 close to the first feeding opening 15 is provided with a baffle plate 19; both a top end and a bottom end of the material groove 14 are equipped with an opening; a bottom opening is provided with a sliding plate 17 assembly; the sliding plate 17 assembly can control opening and closing of the bottom of the material groove 14; the sliding plate 17 assembly includes a sliding plate 17 and a sliding rod 18; the sliding plate 17 is movably connected to the sliding rod 18; the sliding rod 18 is sleeved with a first spring (not shown in the figure); one end of the first spring presses against the sliding plate 17, and the other end presses against the material barrel 8; and when the first spring is in a natural state, the sliding plate 17 seals the bottom of the material groove 14.

Continuing to explain in combination with FIG. 9, the top of the feeder 9 is provided with a second feeding opening 22, and the bottom is provided with a second discharge opening 23; the second discharge opening 23 is provided with a feeding roller 20 and a baffle 21; a tail end of the second discharge opening 23 is provided with the heater 10; the baffle 21 presses against the feeding roller 20 to block the second discharge opening 23; a feeding groove 24 is opened on the feeding roller 20; the heater 10 is open at the top and bottom, the top being a third feeding opening 25, and the bottom being a third discharge opening 26; the bottom of the heater 10 is provided with a switch plate 27; the switch plate 27 is hinged with the heater 10; a torsion spring 28 is arranged at a hinge part; one side of the switch plate 27 extends outward to form a connecting rod 29; when the torsion spring 28 is in a natural state, the switch plate 27 seals the third discharge opening 26; one side of the feeding roller 20 is provided with a transmission assembly; the transmission assembly includes a cam 30, a transmission rod 31, a transmission frame 32 and a micro switch 33; the cam 30 is coaxial with the feeding roller 20; a cam tip 34 and a cam base circle 35 are distributed at 180 degrees; the cam tip 34 is arranged in parallel with the feeding groove 24 with corresponding positions; a contact block 36 is arranged on an outer side of the cam base circle 35; the micro switch 33 is arranged on an outer side of the feeder 9; the transmission rod 31 is in the shape of "n"; the transmission rod 31 is inserted into the transmission frame 32; the top of the transmission rod 31 presses against the cam 30; the transmission rod 31 is sleeved with a second spring 37; one end of the second spring 37 presses against a clamping block of the transmission rod 31, and the other end presses against the transmission frame 32; one vertical rod of the transmission rod 31 extends horizontally outward and presses against the connecting rod 29 of the switch plate 27; during rotation, the feeding roller 20 controls opening and closing of the switch plate 27 of the heater 10 through the transmission assembly; and elasticity of the second spring 37 is larger than elasticity of the torsion spring 28.

During use of the smoker oven provided by this embodiment, the sliding plate 17 on the bottom of the material barrel 8 blocks the bottom of the material groove 14; smoking wood chips are put into the material groove 14; the material barrel 8 is pushed from the material barrel opening 4 into the material barrel cavity 13; during pushing, the baffle plate 19 presses against the sliding plate 17 to push the sliding plate 17 away; the first spring on the sliding rod 18 is in a compressed state; the smoking wood chips in the material groove 14 enter the feeder 9 from the material groove 14 via the first discharge opening 16 and the second feeding opening 22; after the water tank 41 is filled with water, the water tank 41 is put on the water tank seat 40; the water tank seat 40 is pushed from the water tank opening 5 into the water tank cavity 12; the smoking assembly is started; the feeding roller 20 rotates; the smoking wood chips enter the feeding groove 24; when the feeding groove 24 rotates to the lowest point, the smoking wood chips in the feeding groove 24 drop into the heater 10 via the second discharge opening 23 and the third feeding opening 25; the remaining smoking wood chips are stopped by the baffle 21 to still stay in the feeder 9; at this time, the cam tip 34 touches the top of the transmission rod 31 and pushes the transmission rod 31 downward; the second spring 37 is compressed; the torsion spring 28 is in a natural state; the switch plate 27 seals the third discharge opening 26; at the same time, the cam base circle 35 rotates to the topmost part; the contact block 36 touches the micro switch 33; the feeding roller 20 stops rotation; feeding into the heater 10 is stopped, and heating is started; the smoking wood chips after heating generate smoke which is guided by the flue 11 to enter the inner container 2 from the smoke outlet 7 for food smoking; a user can control the amount of smoke of the wood chips by adjusting the temperature of the heater 10; after the heated wood chips are completely burned off, the smoking assembly is started again; the feeding roller 20 rotates again; the cam tip 34 rotates to the topmost part; the second spring 37 is reset to drive the transmission rod 31 to move upward, thereby pushing the connecting rod 29 upward to open the switch plate 27; and the burned-off smoking wood chips drop into the water tank 41 from the fourth feeding opening 38 and are extinguished after touching the water.

The above descriptions are merely the preferred embodiments of the present application, and are not intended to limit the present application. The present application may be subject to various changes and variations to those skilled in the art. Any modification, equivalent substitution, improvement, etc. made within the spirit and principles of the present application should be included within the scope of protection of the present application.

What is claimed is:

1. A smoker oven, comprising a smoking assembly, an inner container and a box body; wherein
the smoking assembly and the inner container are arranged in the box body;
one side of the box body is provided with a box door, a material barrel opening and a water tank opening;
a side of the inner container close to the box door is provided with a cooking opening, and a side of the inner container close to the smoking assembly is provided with a smoke outlet;
the smoking assembly comprises a material barrel, a feeder, a heater, a flue and a water tank cavity;
the feeder is arranged below the material barrel;
a quantitative feeding device is arranged in the feeder;
the heater is arranged below the feeder;
the water tank cavity is arranged below the heater;
a water tank is arranged in the water tank cavity; and
the flue covers above the water tank cavity to communicate the water tank cavity with the smoke outlet.

2. The smoker oven according to claim 1, wherein the quantitative feeding device comprises a feeding roller and a baffle;
a top of the feeder is provided with a second feeding opening;
a bottom of the feeder is provided with a second discharge opening;
the feeding roller and the baffle are arranged at the second discharge opening;
a bottom of the second discharge opening is provided with the heater;
the baffle and the feeding roller press against each other to block the second discharge opening; and
a feeding groove is arranged on the feeding roller.

3. The smoker oven according to claim 2, wherein the heater is provided with a second feeding opening and a third discharge opening; and
the heater is further provided with a discharge device for controlling quantitative drop of wood chips.

4. The smoker oven according to claim 3, wherein
the discharge device is a pushing plug;
the pushing plug is arranged in the heater;
the pushing plug is connected to the feeding roller through an eccentric connecting rod;
rotation of the feeding roller drives the pushing plug to move in the heater; and
a heating sheet is further arranged in the heater.

5. The smoker oven according to claim 3, wherein
the discharge device comprises a transmission assembly and a switch plate;
both a top end and a bottom end of the heater are equipped with an opening;
the switch plate is hinged with the bottom end of the heater;
a torsion spring is arranged at a hinge part;
one side of the switch plate extends outward to form a connecting rod;
one side of the feeding roller is provided with the transmission assembly;
the feeding roller rotates to control opening and closing of the switch plate through the transmission assembly;
the transmission assembly comprises a cam, a transmission rod, a transmission frame and a micro switch;
the cam is coaxial with the feeding roller;
a cam tip corresponds to a position of the feeding groove;
a contact block is arranged on an outer side of a cam base circle;
the micro switch is arranged on an outer side of the feeder;
the transmission rod is inserted into the transmission frame;
a top of the transmission rod presses against the cam;
the transmission rod is sleeved with a second spring;
one end of the second spring presses against the transmission rod, and the other end presses against the transmission frame; and
one end of the transmission rod extends outward and presses against the connecting rod of the switch plate.

6. The smoker oven according to claim 5, wherein the transmission rod is in the shape of "n"; the second spring is sleeved on each vertical rod of the transmission rod; and one vertical rod of the transmission rod extends horizontally outward and presses against the connecting rod of the switch plate.

7. The smoker oven according to claim 1, wherein
the material barrel is arranged in a material barrel cavity;
the material barrel is provided with a material groove;
one side of the material barrel cavity is provided with a first feeding opening;
a bottom of the material barrel cavity is provided with a first discharge opening; and
the first feeding opening and the material barrel opening face each other.

8. The smoker oven according to claim 7, wherein both a top end and a bottom end of the material groove are equipped with an opening; a sliding plate is movably connected at a bottom opening of the material groove; and a baffle plate is arranged on one side of the second feeding opening close to the material barrel opening.

9. The smoker oven according to claim 7, wherein
both a top end and a bottom end of the material groove are equipped with an opening, a sliding plate is movably connected at a bottom opening of the material groove;
a baffle plate is arranged on one side of the first discharge opening close to the first feeding opening;
a bottom of the material barrel is provided with a sliding rod;
the sliding plate is movably connected to the sliding rod;
a first spring is arranged on the sliding rod;
one end of the first spring presses against the sliding plate, and the other end presses against the material barrel; and
when the first spring is in a natural state, the sliding plate seals the bottom of the material groove.

10. The smoker oven according to claim 1, wherein
a top of the water tank cavity is provided with a fourth feeding opening;
one side of the water tank cavity is provided with a fourth discharge opening;
the fourth discharge opening and the water tank opening face each other;
a water tank seat is arranged in the water tank cavity; and
the water tank is arranged in the water tank seat.
